(19) United States
(12) Patent Application Publication
(10) Pub. No.: US 2009/0324718 A1
(43) Pub. Date: Dec. 31, 2009

(54) IMATINIB COMPOSITIONS

(76) Inventors: Ilan Zalit, Rosh Ha Ayin (IL); Boaz Pal, Cambridge, MA (US)

Correspondence Address: KENYON & KENYON LLP, ONE BROADWAY, NEW YORK, NY, 10004, US

(21) Appl. No.: 12/439,582

(22) PCT Filed: Sep. 4, 2007
(86) PCT No.: PCT/US07/19338

§ 371 Date: Jul. 30, 2009

Related U.S. Application Data
(60) Provisional application No. 60/841,707, filed on Sep. 1, 2006.

(51) Int. Cl. A61K 9/28; A61K 31/506

(52) U.S. Cl. 424/474; 514/252.18

(57) ABSTRACT

Provided are compositions of imatinib, methods for their preparation, and methods for treatment using the same.

### CROSS REFERENCE TO RELATED APPLICATIONS

The present application claims the benefit of U.S. Provisional Patent Application No. 60/841,707, filed Sep. 1, 2006, the contents of which is incorporated herein by reference.

### FIELD OF THE INVENTION

The invention relates to compositions of imatinib, methods for their preparation, and methods for treatment using the same.

### BACKGROUND OF THE INVENTION

Imatinib, as exemplified by the mesylate or 4-[(4-Methyl-1-piperazinyl)methyl]-N-[4-methyl-3-[[4-(3-pyridinyl)-2-pyrimidinyl]amino]-phenyl]benzamide methanesulfonate, has the following chemical structure:

It is reported to be a white to off-white to brownish or yellowish tinged

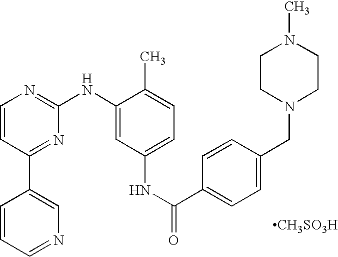

crystalline powder. Imatinib mesylate is understood to be soluble in aqueous buffers having a pH less than or equal to 5.5, but is very slightly soluble to insoluble in neutral/alkaline aqueous buffers. In non-aqueous solvents, the drug substance is apparently freely soluble to very slightly soluble in dimethyl sulfoxide, methanol d ethanol, but is insoluble in n-octanol, acetone and acetonitrile.

Imatinib is indicated for the treatment of newly diagnosed adult patients with Philadelphia chromosome positive chronic myeloid leukemia (CML) in chronic phase.

Imatinib is sold under the brand name Gleevec® (imatinib as mesylate) which is marketed by Novartis Pharmaceuticals. Gleevec® is available in tablets for oral administration in 400 mg and 600 mg strength. The inactive ingredients of Gleevec® are reported to be colloidal silicon dioxide (NF); crospovidone (NF); hydroxypropyl methylcellulose (USP); magnesium stearate (NF); and microcrystalline cellulose (NF). Tablet coating: ferric oxide, red (NF); ferric oxide, yellow (NF); hydroxypropyl methylcellulose (USP); polyethylene glycol (NF) and talc (USP).

Imatinib is generally known to be a hygroscopic material. PCT application WO 2006/048890 describes an “Alpha” form with specific hygroscopic properties, i.e., apparently the water uptake is not more than 1% w/w, preferably not more than 0.6% at 80% RH over a period of 90 hours. Other polymorphs of imatinib have been described including, for example, the mesylate salt. U.S. Pat. No. 6,894,051 describes an allegedly novel non-hygroscopic form of imatinib.

There is a need in the art for stable imatinib compositions in order to achieve the desired therapeutic effect, particularly those that are chemically and physically stable.

### SUMMARY OF THE INVENTION

In one aspect, the present invention provides a solid solution, preferably a stable solid solution, comprising a solid solvent and imatinib selected from amorphous and crystalline forms of imatinib and pharmaceutical acceptable salts 20 thereof. Preferably, imatinib is selected from amorphous and crystalline forms of imatinib mesylate. Preferably, the solid solvent is a polyvinylpyrrolidone (PVP), more preferably Povidone.

In another aspect, the present invention provides a stable pharmaceutical composition comprising a solid solution and at least one pharmaceutically acceptable excipient, wherein the solid solution comprises a solid solvent and imatinib selected from amorphous and crystalline forms of imatinib and pharmaceutical acceptable salts thereof. Preferably, imatinib is selected from amorphous and crystalline forms of imatinib mesylate. Preferably, the solid solvent is a polyvinylpyrrolidone (PVP), more preferably Povidone.

In another aspect, the present invention provides a method of preparing a solid solution of a solid solvent and imatinib, comprising the steps of

a) coprecipitating a mixture comprising imatinib or a pharmaceutically acceptable salt thereof, and a solid solvent from a processing solvent to form a solid solution;

b) optionally combining the solid solution with at least one pharmaceutically acceptable excipient, to form a mixture; and

c) granulating the mixture to form a pharmaceutical composition.

Alternatively, a solid solution of a solid solvent and Imatinib may be prepared by a process comprising:

a) providing imatinib selected from crystalline and amorphous forms of imatinib or pharmaceutical acceptable salts thereof;

b) dissolving a solid solvent in a processing solvent, forming a solution of the solid solvent;

c) mixing imatinib with the solution of the solid solvent, forming a mixture; and

d) removing the processing solvent, forming a solid solution.

In yet another aspect, the present invention provides a method of preparing a pharmaceutical composition comprising a solid solution of a solid solvent and imatinib, comprising the steps of

a) providing imatinib, selected from amorphous and crystalline forms of imatinib and pharmaceutical acceptable salts thereof;

b) mixing imatinib, a solid solvent and a processing solvent, forming a mixture;

c) removing the process solvent, preferably thereby co-precipitating imatinib and a solid solvent from the processing solvent, forming a solid solution of imatinib, preferably by evaporation,

d) optionally mixing the solid solution with at least one pharmaceutical acceptable excipient, forming a solid solution mixture; and

e) granulating the solid solution forming a stable pharmaceutical composition.

The present invention also provides a method of treating a patient suffering from a disease comprising administering to a patient in need thereof a therapeutically effective amount of a stable pharmaceutical composition comprising a solid solution, wherein the solid solution comprises a solid solvent and imatinib selected from amorphous and crystalline forms of imatinib and pharmaceutical acceptable salts thereof. Preferably, imatinib is selected from amorphous and crystalline forms of imatinib mesylate.

### BRIEF DESCRIPTION OF THE FIGURES

FIG. 1. Shows the morphology of imatinib particles in a solid solution of PVP, at a ratio of imatinib:PVP of 1:0.5 (wt/wt)

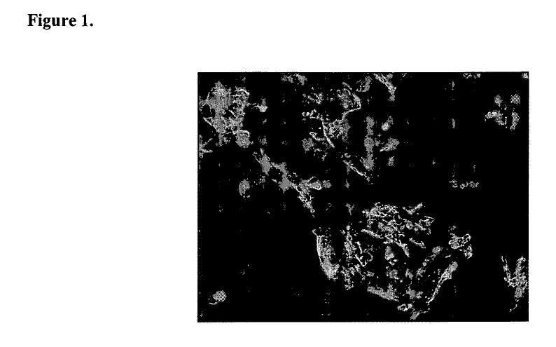

FIG. 2. Shows the morphology of imatinib particles in a solid solution of PVP, at a ratio of imatinib:PVP of 1:2 (wt/wt).

FIG. 3. First row: Slugs prepared according to Example 5 after storage for 28 days at 40° C. and 75% relative humidity, stored (a) without silica gel inserts or (b) with silica gel inserts. Second row: crystalline raw 10 material after storage for 28 days at 40° C. and 75% relative humidity, stored (a) without silica gel inserts or (b) with silica gel inserts.

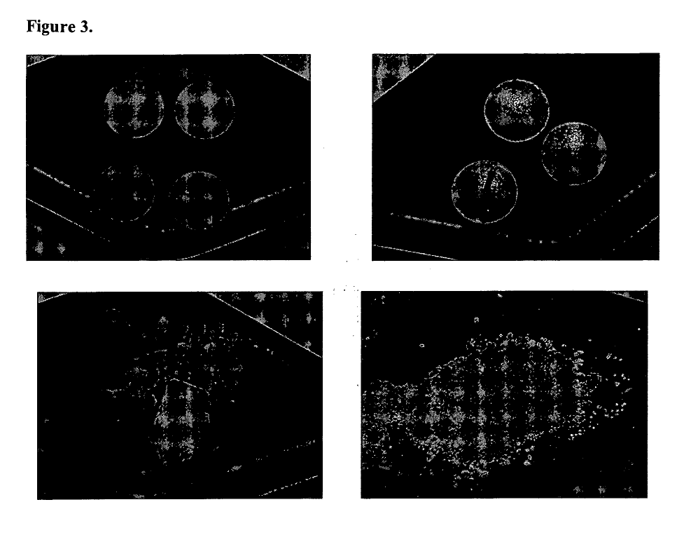

### DETAILED DESCRIPTION OF THE INVENTION

As used herein the term “room temperature” refers to the ambient temperature of an typical laboratory, which is usually about that of Standard Temperature and Pressure (STP).

The term “solid solvent” as used herein describes a solid carrier that is capable of forming a solid solution with one or more additional solids described herein. A “solid solution” is a homogeneous solid that can exist over a range of component chemicals.

The term “stable” as used herein relates to a substance which is chemically and/or physically stable.

The term “chemical stability” as used herein relates to the presence or absence of degradation products of the active pharmaceutical ingredient (API) as measure over time. Chemical stability is measured as the Assay of the material and/or the level of degradants. Stability is defined as having an Assay of at least about 90%, as determined by an HPLC assay method and/or having a minimum level of degradants, as determined by an HPLC Impurities and Degradation Determination (IDD) method over time. Preferably, a chemically stable composition as in the present inventor has an assay of at least 95%, more preferably at least 98%, as determined by an HPLC assay over time (storage time).

As used herein the term “physical stability” of a composition means the appearance of the composition is substantially unchanged and/or the hygroscopicity is low.

The term “appearance” as used herein describes the color and texture of a composition. The color of a composition according to a scored scale, as assessed by one of ordinary skill in the art in view of the following guidelines. A discoloration score of 1 indicates material without discoloration, whereas a discoloration score of 5 indicates severe discoloration (almost complete discoloration) relative to the color of the material as obtained after preparation and prior to storage. In the present invention the composition preferably has a white color immediately after processing and generally remains white if stable. Discoloration to a yellow color is typical when the composition is unstable. Similarly, a texture score of 1 indicates material having an uniform and smooth texture, whereas a texture score of 5 indicates a nonuniform and rough texture. The texture may be determined by visual inspection by one of ordinary skill in the art. For both discoloration and texture, scores 2 to 4 represent 5 “some or slight (change)” for a score of 2, “medium (change)” for a score of 3, and “substantial (change)” for a score of 4.

Texture is often highly correlated with hygroscopicity. Water/moisture absorption tends to reduce shine, for example. Water hygroscopicity can be determined by weight gain analyses.

Preferred embodiments of the invention have the following stability characteristics: (a) a coloration score of 4 or less and/or 3 or less and/or 2 or less, and/or a texture score of 3 or less and/or 2 or less after storage (i) at a temperature of about 55° C. and about 75% relative humidity for 5 days, and/or (ii) at a temperature of about 40° C. and about 75% relative humidity for 30 days. Preferably, a solid solution of the invention has a coloration score of 3 or less, and preferably a pharmaceutical composition of the invention has a coloration score of 4 or less.

It was determined that the physical state, in particular hygroscopicity, of imatinib depends to a certain degree on the physical structure of the active drug, e.g. its polymorphism. As an example, amorphous API material tended to be much more hygroscopic when compared to crystalline material. It is known in the art that hygroscopic active materials will sometimes tend to be chemically and physically unstable as compared to the corresponding crystalline material. Therefore, in one embodiment the present invention provides processes and compositions that will enable development and/or manufacture imatinib formulations where the physical state of the active drug has less effect on the product's chemical stability and/or physical stability.

Stability of an active pharmaceutical ingredient such as imatinib in the present invention may be improved, for example, by processes that use stabilized solid solutions of imatinib as a “drug source” in imatinib formulations; that use a different granulation process; or both. A product can be further optimized using packaging materials such as dessicants.

In one aspect, the present invention provides a solid solution comprising a solid solvent and imatinib selected from amorphous and crystalline forms of imatinib and pharmaceutical acceptable salts thereof. Preferably, the solid solution is a stable solution. Preferably, imatinib is selected from amorphous and crystalline forms of imatinib mesylate. Preferably, the solid solvent is a polyvinylpyrrolidone (PVP), more preferably Povidone.

A preferred solid solution of the present invention provides greater stability in solutions comprising at least about 50% of the solid solvent by weight. Accordingly, solid solutions comprising imatinib of the present invention may have a weight/weight ratio of imatinib:Solid Solvent (preferably imatinib:Povidone) in the range from about 1:0.17 to about 1:4, preferably about 1:0.5 to about 1:4, more preferably from about 1:1 to about 1:2.

In another aspect, the present invention provides a stable pharmaceutical composition comprising a solid solution and at least one pharmaceutically acceptable excipient, wherein the solid solution comprises a solid solvent and imatinib selected from amorphous and crystalline forms of imatinib and pharmaceutical acceptable salts thereof. Preferably, the solid solvent is a polyvinylpyrrolidone (PVP), more preferably the solid solvent is Povidone.

In another embodiment of the present invention there is provided a stable pharmaceutical composition comprising a solid solution of a solid solvent and imatinib, which has at least one pharmaceutically acceptable excipient. The pharmaceutically acceptable excipient is selected from the group consisting of tablet and capsule fillers and diluents (such as microcrystalline cellulose, lactose, starch and tri-basic calcium phosphate), disintegrants (such as starch, croscarmellose sodium, crospovidone and sodium starch glycolate), and glidants (such as colloidal silicon dioxide and talc), lubricants (such as magnesium stearate, sodium lauryl sulfate, stearic acid and sodium stearyl fumarate), binders (such as starch and pregelatinized starch).

More particularly, suitable diluents and fillers for use in the pharmaceutical composition of the present invention include microcrystalline cellulose (e.g. Avicel®), lactose, starch, pregelatinized starch, calcium carbonate, calcium sulfate, sugar, dextrates, dextrin, dextrose, dibasic calcium phosphate dihydrate, tribasic calcium phosphate, magnesium carbonate, magnesium oxide, maltodextrin, mannitol, powdered cellulose, sodium chloride, sorbitol and talc.

Solid pharmaceutical compositions of the present invention that are compacted into a dosage form, such as a tablet, may include the addition of a disintegrant to the composition. Disintegrants include croscarmellose sodium (e.g. Ac Di Sol®, Primellose®), crospovidone (e.g. Kollidon®, Polyplasdone®), microcrystalline cellulose, polacrilin potassium, powdered cellulose, pregelatinized starch, sodium starch glycolate (e.g. Explotab®, Primoljel®) and starch.

Glidants can be added to improve the flowability of a solid composition before compaction and to improve the accuracy of dosing especially during compaction and capsule filling. Excipients that may function as glidants include colloidal silicon dioxide, magnesium trisilicate, powdered cellulose, and talc.

A lubricant can be added to the composition to reduce adhesion and/or ease the release of the product from e.g. the dye. Lubricants include magnesium stearate calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc and zinc stearate.

Binders may be incorporated into the formulation. Binders are typically utilized if the manufacture of the dosage form uses a granulation step. Examples of suitable binders include povidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch. Also, binders are often the same polymers as the polymers used to control the release of the active ingredient from the formulation.

Other excipients that may be incorporated into the formulation include preservatives, surfactants, antioxidants, or any other excipient commonly used in the pharmaceutical industry. An optional tablet coat is preferably cosmetic and may be prepared from, for example, commercially available powders for coating suspensions based on either Hypromellose or Polyvinyl alcohol, together with polyethylene Glycol and colorants etc.

In a preferred embodiment of the present invention, the stable pharmaceutical composition comprises in addition to a solid solution of a solid solvent and imatinib, Croscarmellose sodium, Pregelatinized starch (1500), Lactose, and Magnesium Stearate.

The solid stable pharmaceutical compositions of the present invention include powders, granulates, aggregates and compacted compositions. The dosages include dosages suitable for oral, buccal, and rectal administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts. Preferably, the dosage form comprises about 50 mg to about 500 mg imatinib, more preferably about 100 mg to about 400 mg imatinib.

The pharmaceutical composition of the present invention may be prepared in any dosage form such as a compressed granulate in the form of a tablet for example. Also, uncompressed granulates and powder mixes that are obtained by the method of the present invention in the pre-compression steps can be simply provided in a dosage form of a capsule or sachet. Therefore, dosage forms of the pharmaceutical 20 composition of the present invention include solid dosage forms like tablets, powders, capsules, sachets, etc. The dosage form of the present invention may also be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

Embodiments of the invention are preferably packaged with a desiccant, such as silica. The container preferably has a high moisture barrier, examples of which are known in the art. In a preferred packaged pharmaceutical composition of the present invention an amount of desiccant which will assure a weight gain due to moisture of not more than (NMT) 5%, preferably 0.5%, more preferably 0.05%, is preferred in order to maintain a good appearance and low water absorption for a maximum period of time. For example, the weight gain of the packaged material of the present invention after storage (i) at a temperature of about 55° C. and about 75% relative humidity for 5 days, and/or (ii) at a temperature of about 40° C. and about 75% relative humidity for 30 days, is less than about 15%, preferably less than about 10%, more preferably less than about 5% by weight.

In another aspect, the present invention provides a method of preparing a solid solution of a solid solvent and imatinib, comprising the following steps of

a) providing imatinib selected from crystalline and amorphous forms of imatinib or pharmaceutical acceptable salts thereof;

b) dissolving a solid solvent in a processing solvent, forming a solution of the solid solvent;

c) mixing imatinib with the solution of the solid solvent, forming a mixture and

d) removing the processing solvent, forming a solid solution.

In the method of preparing a solid solution of the present invention the solid solvent is preferably a polyvinylpyrrolidone (PVP), more preferably Povidone. A preferred imatinib is selected from crystalline and amorphous forms of imatinib mesylate. Further, the processing solvent in the method of the present invention preparing a solid solution is an organic solvent, preferably a C₁-C₄ alcohol. Removal of the processing solvent may be through any suitable process available in forming a solid solution. A preferred process of removing the processing solvent is by evaporation of the processing solvent. Preferably removal of the processing solvent in the method of the present invention results in the co-precipitation of imatinib (or a pharmaceutically acceptable salt thereof) and the solid solvent.

An exemplary solid solution of imatinib may be prepared by dissolving imatinib mesylate and a solid solvent (e.g. PVP) in a processing solvent, e.g., ethanol, followed by evaporation of the processing solvent. This product may be prepared from any source (crystalline or amorphous) of imatinib or a pharmaceutical acceptable salt thereof. Preferred solid solutions have improved stability when compared to free drug amorphous API material, even though the imatinib within the solid solution might be described as being in an amorphous state. (See, e.g., table 2 example 2 compared to Active drug amorphous). This improvement depends at least to a certain degree on the ratio of imatinib (or a pharmaceutical salt thereof) to solid solvent (e.g., povidone) in the final solid solution. In preferred embodiments, increasing the content of solid solvent in the solid solution increases the physical stability of the composition. Accordingly, solid solutions of a solid solvent and imatinib are preferably present in a ratio of imatinib (or a pharmaceutically acceptable salt thereof):Solid Solvent (preferably imatinib:Povidone) in the range from about 1:0.17 to about 1:4, preferably from about 1:0.5 to about 1:4, more preferably from about 1:1 to about 1:2 (wt:wt). The solid solution product has improved flow properties and therefore could be directly compressed into tablet or may be processed by other conventional means.

In another aspect, the present invention provides a method of preparing a pharmaceutical composition comprising a solid solution of a solid solvent and imatinib, comprising the following steps of a) providing a solid solution of a solid solvent and imatinib, selected from amorphous and crystalline forms of imatinib and pharmaceutical acceptable salts thereof;

b) mixing the solid solution with at least one pharmaceutical acceptable excipient, forming a solid solution mixture; and

c) processing the solid solution mixture to form a pharmaceutical composition.

The processing of the solid solution mixture may be any suitable process available in forming a pharmaceutical composition from a mixture of an active pharmaceutical ingredient and at least one pharmaceutical excipient, preferably processing comprises granulation or direct compression of such mixture.

In preparing a pharmaceutical composition of the present invention a granulation process involves mixing the solid solution comprising imatinib and at least one pharmaceutical excipient in a mixer. In one embodiment, a granulating solvent, solution or suspension is added to the dry powders in the mixer and mixed until the desired characteristics are achieved. This usually produces a granule that will be of suitable characteristics for producing tablets with adequate hardness, dissolution, content uniformity, and other physical characteristics. After the wet granulation step, the product is most often dried and then milled after drying, to obtain a major percentage of the product within a desired size range. Preferably, the product after wet granulation is dried until the loss on drying (LOD) is not more than about 1.5%, more preferably not more than about 1.1%. Preferably, the product is milled or sized through an 1 mm screen, more preferably through a 0.8 mm screen. In a dry granulation process (also referred to as slugging) for preparing a pharmaceutical composition comprising the solid solution of the present invention and at least one pharmaceutical excipient, a mixture is prepared as above in a mixer without the addition of a granulating solvent.

A preferred formulated solid solution has improved physical stability when compared to conventional wet granulation formulation, or to a formulation made from amorphous material. See, e.g., table 1, example 4 (solid solution) compared to example 9 (amorphous material) or compared to example 7 (crystalline material—wet granulation with water). In addition, due to the high percentage of the active drug in a preferred embodiment of the formulated product (up to about 50% by weight) the physical properties of the active drug including flow properties, a direct compression process to prepare a pharmaceutical composition of the present invention is less preferred in the manufacture process of imatinib tablets when using free drug imatinib. Under these circumstances, dry processing, e.g., a dry granulation process (Table 2 P-00693) or wet granulation with an organic solvent (e.g. EtOH) (Table 2; P-00695) is preferred to wet granulation with water (Table 2; P-00694). A process of preparing a stable pharmaceutical composition of the present invention that avoids the use of water (dry granulation, direct compression, or wet granulation with a C₁-C₄ alcohol, preferably ethanol) is therefore preferred over a similar process using water (wet granulation with water). In a preferred method of the present invention, the stable pharmaceutical composition of the present invention is preferably prepared by dry granulation or by wet granulation with a suitable granulating solvent. A suitable granulating solvent is an organic solvent. More preferably, the granulating solvent is a C₁-C₄ alcohol or combinations thereof.

The method of the present invention may further comprise steps in preparing a tablet of the pharmaceutical composition of the present invention. In preparing such tablet the step of processing the solid solution mixture to form a pharmaceutical 25 composition comprises the steps of

a) mixing the solid solution with one or more excipients forming a final blend;

b) pressing the final blend into a tablet; and

c) optionally coating the tablet with a cosmetic coat.

Capsules comprising either a hard or soft shell and containing the composition of the present invention may be prepared. The shell may be made from gelatin and optionally contain a plasticizer such as glycerin and sorbitol, and an opacifying agent or colorant. A capsule filling of the present invention may comprise the granulates that were described with reference to tableting, a final blend of a granulate composition of the present invention mixed with one or more excipients, however they are not subjected to a final tableting step. Further, such capsules may be prepared by any of the methods well known in the pharmaceutical arts.

The present invention also provides a method of treating a patient suffering from a disease comprising administering to a patient in need thereof a therapeutically effective amount of a stable pharmaceutical composition comprising a solid solution, wherein the solid solution comprises a solid solvent and imatinib selected from amorphous and crystalline forms of imatinib and pharmaceutical acceptable salts thereof. Preferably, imatinib is selected from amorphous and crystalline forms of imatinib mesylate. Preferably, the method of treatment of the present invention is treating a patient suffering from Philadelphia chromosome positive myeloid leukemia.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to limit its scope in any way.

### EXAMPLES

The stability of the examples described herein were tested using various analytical methods. One analytic method used is a determination using HPLC. The following HPLC method was used for the examples described below where HPLC was used as an analytic method.

HPLC METHOD

| | |
|---|---|
| Column type | hypersil C18, BDS, 5μ, 4.6*150 mm |
| Column no. | 23a |
| Column temperature | RT (room temperature) |
| Mobile phase program | 30 mM sodium heptane sulphonic |
| | acid in 0.01M KH₂PO₄ (pH 2.5): |
| | MeOH (42:58) |
| Flow (ml/min) | 0.8 |
| Injection volume (μl) | 20 |
| Injector wash solution | Methanol |
| Detector and wave | UV at 237 nm for imatinib |
| length | |
| Run time (min) | 10 |
| Sample prep. | Equivalent to 100 mg imatinib |
| | (formulated/none formulated) into |
| | 50 mL of mobile phase. |

### Example 1

### Preparation of Imatinib Solid Solution

A 1:1 (wt/wt) solid solution of imatinib mesylate in Povidone was prepared by dissolving Povidone in ethanol and adding imatinib mesylate to the resultant solution/mixture. The ethanol is evaporated to produce a solid solution of imatinib in Povidone.

### Manufacturing Procedure

1) 1 gram of povidone (PVP K-30), was dissolved in 100 grams Ethanol in an appropriate vessel.
2) 1 gram of imatinib mesylate raw material was dissolved into the solution resultant from step 1.
3) The solvent was evaporated from the solution using rotor-vaporization for 30-50 min (at temperature of 55° C.).
4) Obtained solid solution was collected from vessel.

### Example 2

### Preparation of Imatinib Solid Solution

A 1:2 solid solution of imatinib mesylate in Povidone was prepared by dissolving Povidone in ethanol and adding imatinib mesylate to the resultant 20 solution/mixture. The ethanol is evaporated to produce a solid solution of imatinib mesylate in Povidone.

### Manufacturing Procedure

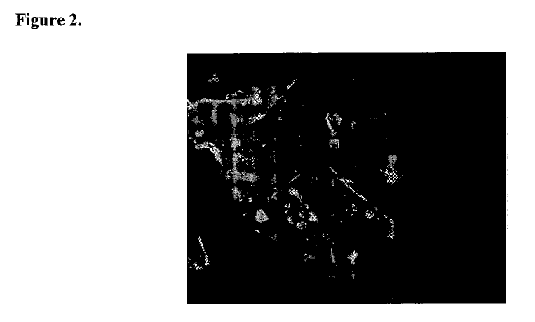

1) 2 grams of povidone (PVP K-30), was dissolved in 100 grams Ethanol in an appropriate vessel.
2) 1 gram of imatinib mesylate raw material was dissolved into the solution resultant from step 1.
3) The solvent was evaporated from the solution using rotor-vaporization for 30-50 min (at temperature of 55° C.).
4) Obtained solid solution was collected from vessel (FIG. 2).

Samples of imatinib solid solutions as prepared in Examples 1 and 2 were tested for chemical and physical stability. The samples were tested “as is” or formulated (Examples 3 and 4).

### Example 3

### Formulation Comprising Solid Solution of Imatinib (imatinib:PVP 1:0.5)

A formulation was prepared containing the following excipients and a solid solution of imatinib mesylate (imatinib:PVP 1:0.5). The formulation was prepared by direct compression of a mixture of the following materials.

| | Amount | |
|---|---|---|
| Raw material | (mg/dose) | % |
| imatinib mesylate (solid | 150.0 | 60.0 |
| sol. 1:0.5) | | |
| Croscamellose sodium | 18.0 | 7.2 |
| Pregelatinized Starch (1500) | 45.0 | 18.0 |
| Lactose | 35.0 | 14.0 |
| Magnesium stearate | 2.0 | 0.8 |
| Theoretical end weight | 250 | 100.0 |

### Example 4

### Formulation Comprising Solid Solution of Imatinib (imatinib:PVP 1:1)

A formulation was prepared containing the following excipients and a solid solution of imatinib mesylate. The imatinib mesylate solid solution of example 1 was used of imatinib:PVP (1:1). The formulation was prepared by direct compression of a mixture of the following materials.

| | Amount | |
|---|---|---|
| Raw material | (mg/dose) | % |
| imatinib mesylate solid | 200.0 | 66.7 |
| solution | | |
| Croscamellose sodium | 18.0 | 6 |
| Pregelatinized Starch (1500) | 45.0 | 15 |
| Lactose | 35.0 | 11.67 |
| Magnesium stearate | 2.0 | 0.67 |
| Theoretical end weight | 300 | 100 |

### Examples 5-8

### Testing of Different Formulation Methods

The effect of different formulation techniques on the stability was tested on compositions comprising a solid solution of imatinib in the solid solvent Povidone

| Example | |
|---|---|
| no | Description |
| 5 | Tablets, based on Slugs - dry Granulation |
| 6 | Tablets, by direct compression |
| 7 | Tablets, based on wet granulation with water |
| 8 | Tablets, based on wet granulation with ethanol |

Examples 5-8 were prepared using the following ingredients based on various techniques described in the table above, slugging; compression; granulation etc:

| | Amount | |
|---|---|---|
| | (mg/dose) | % |
| Imatinib mesylate | 100.00 | 50% |
| PVP K-30 | 17.00 | 8.5% |
| Ac-Di- Sol | 10.00 | 5% |
| Starch 1500 | 35.00 | 17.5% |
| Lactose | 35.00 | 17.5% |
| Magnesium stearate | 3.00 | 1.5% |
| Theoretical end weight | 200 | 100 |

Where granulation was involved at least a portion of the Magnesium Stearate was added extra-granularly, in the case of dry granulation with slugs, the Magnesium Stearate was added in two stages, at the slugging state and extra-granularly before tabletting.

### Example 9-10

### Comparison of Formulations with Different Forms of Imatinib in the Solid Solution

Examples 9-10 were prepared using the same formulation and process as described for example 6 above. Examples 9 and 10 however are using as the active pharmaceutical ingredient (API) imatinib having a different morphology. In example 9 amorphous material and in example 10 another crystalline material are used.

The table below summarizes all test results (Assay, IDD, weight gain, and appearance). The appearance data is scored from 1 to 5 (1=very good (no change in color and texture of the sample), see an example of “Score=1” in Table 2, example 10, Day 0, 5=not good (changed both in color (became yellow) and in texture (absorbed water and reduced shine of sample)), see an example of “Score=4-5” in Table 2, example 9, time Day 5), and is supported by pictures presented in Table 2.

TABLE 1
Results Summary table of physical, chemical and appearance tests.

| | | 55° C./75% RH Day 0 | | | | | | |
|---|---|---|---|---|---|---|---|---|
| | | Appearance | Physical | | | | | |
| | | Appearance | | | | | | |
| | | scale: | | Chemical | | | | |
| | | 1 = good | | | main | | 55° C./75% RH Day 5 | |
| Ex. no | Description | 5 = bad | | Assay | deg | total | Appearance | Physical |
| | Active drug (Crystalline 1) | 1 | Alpha | 97.5 | 0.12 | 0.12 | 1 | |
| | Active drug (Crystalline 2) | 1 | | 94.5 | 0.11 | 0.20 | 4 | |
| | Active drug (Crystalline 2) | | | | | | | |
| | Packed with silica | | | | | | | |
| | Active drug (Amorphous) | 1 | amrph | 88.6 | 0.46 | 0.46 | 5 | |
| 1 | Solid solution | 1 | | 93.2 | 0.37 | 0.37 | 1 | |
| | (PVP:Active amorphous* | | | | | | | |
| | 1:1) | | | | | | | |
| 2 | Solid solution | 1 | | 92.1 | 0.21 | 0.21 | 1 | |
| | (PVP:Active amorphous* | | | | | | | |
| | 1:2) | | | | | | | |
| 10 | Tabs - Dry mix | 1 | Alpha | 93.9 | 0.11 | 0.11 | 1-2 | |
| | (Crystaline1) | | | | | | | |
| 6 | Tabs - Dry mix | 1 | | 92.1 | 0.12 | 0.2 | 3-4 | |
| | (Crystaline2) | | | | | | | |
| 9 | Tabs - Dry Mix | 1 | amrph | 89.9 | 0.46 | 0.46 | 4-5 | |
| | (Amorphous) | | | | | | | |
| 3 | Tabs - Solid solution | 1 | amrph | 93.7 | 0.29 | 0.43 | 3-4 | |
| | (PVP:Active amorphous* | | | | | | | |
| | 1:0.5) | | | | | | | |
| 4 | Tabs - Solid solution | 1 | | 87.9 | 0.36 | 0.36 | 3-4 | |
| | (PVP:Active amorphous* | | | | | | | |
| | 1:1) | | | | | | | |
| 5 | Tabs - Slug | 1 | | 93.5 | <0.01 | <0.01 | 4-5 | |
| | (Crystalline 2) | | | | | | | |
| | Tabs - Slug (Crystalline 2) | | | | | | | |
| | Packed with silica | | | | | | | |
| 7 | Tabs - Wet/Water | 1 | | 94.4 | <0.01 | <0.01 | 5 | |
| | (Crystalline 2) | | | | | | | |
| 8 | Tabs - Wet/EtOH | 1 | | 85.5 | 0.13 | 0.13 | 3-4 | |
| | (Crystalline 2) | | | | | | | |

| | | | | 40° C./75% RH | | | | | |
|---|---|---|---|---|---|---|---|---|---|
| | | | | Gain of weight (%) | | | | | |
| | | | | Crystalline 2; RM (t0) = 107 | | | | | |
| | | | | Crystalline 2; RM + silica (t0) = 107 | | | | | |
| | | | | Crystalline 2; slug (4 tabs) (t0) = 784.6 | | | | | |
| | 55° C./75% RH Day 5 | | | Crystalline 2; slug (4 tabs) + silica | | | | | |
| | Chemical | | | (t0) = 797.15 | | | | | |
| | | Main | | Appearance | Wt gain | Appearance | Wt gain | Appearance | Wt gain |
| Ex. no | Assay | deg | Total | t = 7 days | T = 7 days | t = 14 days | T = 14 days | t = 28 days | T = 28 days |
| | 98.8 | 0.12 | 0.12 | | | | | | |
| | 98.8 | 0.13 | 0.22 | 1 | 21.49 | 1-2 | 17.75 | 3 | 18.3 |
| | | | | 1 | 12.42 | 1 | 7.79 | 2 | 8.86 |
| | 97.8 | 0.44 | 0.44 | | | | | | |
| 1 | 88.7 | 0.36 | 0.36 | | | | | | |
| 2 | 88.1 | 0.2 | 0.2 | | | | | | |
| 10 | 91.5 | 0.11 | 0.11 | | | | | | |
| 6 | 88 | 0.14 | 0.24 | | | | | | |
| 9 | 91.4 | 0.47 | 0.47 | | | | | | |
| 3 | 90.22 | 0.4 | 0.57 | | | | | | |
| 4 | 83.8 | 0.36 | 0.36 | | | | | | |
| 5 | 92.6 | 0.25 | 0.25 | 1 | 1.68 | 2 | 1.96 | 3-4 | 2.55 |
| | | | | 1 | −3.54 | 1 | −3.38 | 2 | −3.11 |
| 7 | 96.9 | 0.32 | 0.67 | | | | | | |
| 8 | 87.9 | 0.13 | 0.13 | | | | | | |

1. A solid solution comprising imatinib and a solid solvent.

2. The solid solution of claim 1, wherein the solid solution is stable.

3. The solid solution of claim 1, wherein the imatinib is selected from amorphous and crystalline forms of imatinib and pharmaceutical acceptable salts thereof.

4. The solid solution of claim 3, wherein the imatinib is selected from amorphous and crystalline imatinib mesylate.

5. The solid solution of claim 1, wherein the solid solvent is polyvinylpyrrolidone (PVP).

6. The solid solution of claim 5, wherein the solid solvent is Povidone.

7. The solid solution of claim 1, wherein the ratio of imatinib to solid solvent is in the range from 1:0.17 to 1:4 (wt/wt).

8. The solid solution of claim 7, wherein the ratio of imatinib to solid solvent is in the range from 1:0.5 to 1:2 (wt/wt).

9. The solid solution of claim 8, wherein the ratio of imatinib to solid solvent is 1:2 (wt/wt).

10. The solid solution of claim 1, wherein the solid solution has an color score of 4 or less after storage at 55° C./75% relative humidity for seven days or at 40° C./75% relative humidity for 14 days.

11. The solid solution of claim 10, wherein the color score is 3 or less.

12. The solid solution of claim 11, wherein the color score is 2 or less.

13. A pharmaceutical composition comprising a solid solution in accordance with claim 1.

14. The pharmaceutical composition of claim 13, wherein the pharmaceutical composition has an color score of 4 or less upon storage at 55° C./75% relative humidity for seven days or at 40° C./75% relative humidity for 14 days.

15. The pharmaceutical composition of claim 14, wherein the color score is 3 or less.

16. A method of preparing a solid solution of a solid solvent and imatinib, comprising the following steps of
a) providing imatinib, selected from amorphous and crystalline forms of imatinib and pharmaceutical acceptable salts thereof;
b) dissolving a solid solvent in a processing solvent, forming a solution of the solid solvent;
c) mixing imatinib with the solution of the solid solvent, forming a mixture; and
d) removing the processing solvent, forming a solid solution.

17. The method of preparing a solid solution of claim 16, wherein the imatinib is selected from amorphous and crystalline forms of imatinib and pharmaceutical acceptable salts thereof.

18. The method of preparing a solid solution of claim 17, wherein the imatinib is selected from amorphous and crystalline imatinib mesylate.

19. The method of preparing a solid solution of claim 16, wherein the solid solvent is polyvinylpyrrolidone (PVP).

20. The method of preparing a solid solution of claim 19, wherein the solid solvent is Povidone.

21. The method of preparing a solid solution of claim 16, wherein the processing solvent is a C₁-C₄ alcohol.

22. The method of preparing a solid solution of claim 21, wherein the processing solvent is ethanol.

23. The method of preparing a solid solution of claim 16, wherein removing the processing solvent comprises evaporating the processing solvent.

24. The method of preparing a solid solution of claim 16, wherein removing the processing solvent comprises co-precipitation of imatinib and the solid solvent from the processing solvent.

25. The method of preparing a solid solution of claim 24, wherein the co-precipitation is carried out by evaporation of the processing solvent.

26. A method of preparing a pharmaceutical composition comprising a solid solution of a solid solvent and imatinib, comprising the steps of;
a) providing a solid solution comprising a solid solvent and imatinib, selected from amorphous and crystalline forms of imatinib and pharmaceutical acceptable salts thereof in accordance with claim 16;
b) mixing the solid solution with at least one pharmaceutical acceptable excipient, forming a solid solution mixture; and
c) processing the solid solution mixture to form a pharmaceutical composition.

27. The method of preparing a pharmaceutical composition of claim 26, wherein step c) comprises granulating the solid solution mixture forming a stable pharmaceutical composition.

28. The method of preparing a pharmaceutical composition of claim 27, wherein granulating the solid solution mixture does not involve the use of water as a granulating solvent.

29. The method of preparing a pharmaceutical composition of claim 26, further comprising;
a) mixing the composition with one or more excipients forming a final blend; and
b) pressing the final blend into a tablet.

30. The method of preparing a pharmaceutical composition of claim 29, further comprising coating the tablet with a cosmetic coat.

31. A method of treating a patient suffering from a disease comprising administering to a patient in need thereof a therapeutically effective amount of a stable pharmaceutical composition comprising a solid solution, wherein the solid solution comprises a solid solvent and imatinib selected from amorphous and crystalline forms of imatinib and pharmaceutical acceptable salts thereof.

32. The method of treating a patient of claim 31, wherein the patient is suffering from Philadelphia chromosome positive myeloid leukemia.

33-34. (canceled)
